(19) United States
(12) Patent Application Publication
(10) Pub. No.: US 2011/0189781 A1
(43) Pub. Date: Aug. 4, 2011

(54) DISTANCE-CONTROLLED ENERGY TRANSFER DYE COMPLEXES

(76) Inventors: Peter Klauth, Monchengladbach (DE); Manfred Rietz, Erkelenz (DE)

(21) Appl. No.: 13/001,504

(22) PCT Filed: Apr. 28, 2009
(86) PCT No.: PCT/EP2009/003067

§ 371 Date: Apr. 18, 2011

(30) Foreign Application Priority Data
Jun. 26, 2008 (DE) 10 2008 029 936.7
Jul. 18, 2008 (DE) 10 2008 033 871.0

(51) Int. Cl. G01N 33/52

(52) U.S. Cl. 436/94

(57) ABSTRACT

The invention relates to the use of at least two different fluorophores for configuring a fluorescence resonance energy transfer pair (FRET pair), wherein at least one first fluorophore (A) serves as the donor fluorophore and at least one second fluorophore (B) serves as the acceptor fluorophore within the FRET pair, wherein the first fluorophore (A) and the second fluorophore (B), independently of each other, each are configured on the basis of an organo-metal complex of rare earth elements, wherein the fluorophores (A) and (B) comprise different rare earth elements from each other.

### CROSS-REFERENCES TO RELATED APPLICATIONS

This application is a National Stage filing of International Application PCT/EP 2009/003067, filed Apr. 28, 2009, entitled “DISTANCE-CONTROLLED ENERGY TRANSFER DYE COMPLEXES” claiming priority to German Applications No. DE 10 2008 029 936.7 filed Jun. 26, 2008, and DE 10 2008 033 871.0 filed Jul. 18, 2008. The subject application claims priority to PCT/EP 2009/003067, and to German Applications No. DE 10 2008 029 936.7 and DE 10 2008 033 871.0 and incorporates all by reference herein, in their entirety.

### BACKGROUND OF THE INVENTION

The present invention relates to the use of at least two different fluorophores for configuring a fluorescence resonance energy transfer pair (FRET pair).

In addition, the present invention relates to a fluorescence resonance energy transfer complex (FRET complex) that comprises at least a first fluorophore (A) as a donor fluorophore and at least a second fluorophore (B) as an acceptor fluorophore as well as a special organic radical joining the fluorophores (A) and (B).

The present invention further relates to a fluorescence resonance energy transfer system (FRET system).

In addition, the present invention relates to the use in accordance with the invention of the FRET complex or FRET system according to the invention for various purposes, in particular for detection of an event in a sample, or for interaction with a target molecule or a target structure, and/or for detection or identification or for determination of a target molecule or a target structure.

Furthermore, the present invention relates to the use of the FRET complex or FRET system according to the invention for further applications, for example as a dye, in particular a fluorescence dye, or else as, or in biosensors or as, or in (bio)probes, in particular FRET probes.

Lastly, the present invention relates to a fluorescence resonance energy transfer pair (FRET pair) that comprises at least two different fluorophores, within the FRET pair at least a first fluorophore (A) acting as a donor fluorophore and at least a second fluorophore (B) acting as an acceptor fluorophore.

Organo-metal rare earth complexes, in particular the β-diketonates and aromatic carboxylates thereof, have already found applications in various fields in which their unique luminescence mechanism (or ‘antenna effect’) is advantageous, for example in biosensors.

Fluorescence resonance energy transfer (FRET) is a physical process known in the prior art, in which energy of an excited fluorescence dye (also referred to as a donor or donor fluorophore) generally can be transferred to a second fluorescence dye (also synonymously referred to as an acceptor or acceptor fluorophore or quencher). The intensity of the energy transfer or FRET depends particularly on the distance between the two fluorophores. The underlying principle of FRET is that, between two dyes that are capable of fluorescence, the energy of an excited donor fluorophore can be transferred to an acceptor fluorophore, this energy transfer to the fluorescing acceptor not occurring in the form of fluorescence, but in a radiationless manner, for example via dipole/dipole mutual reactions.

In order to enable a radiationless energy transfer of this type to the acceptor using FRET, various criteria must be satisfied. On the one hand, the emission spectrum of the donor should overlap with the absorption spectrum of the acceptor. On the other hand, the donor on the one hand and the acceptor on the other should have parallel electronic planes of polarisation, and lastly the donor and the acceptor should be distanced from one another by only a nanometre or a few nanometres since the intensity of the FRET signal decreases with the sixth power of the distance between the two fluorophores. Since the intensity of FRET depends, inter alia, on the distance between the two fluorophores, the use of FRET has become established to a certain extent as an optical nanometre scale, particularly in biochemistry and cell biology, it being possible in this connection to detect protein/protein, protein/nucleic acid and nucleic acid/nucleic acid mutual reactions in particular, and spatial structural changes, such as conformational changes, in biomolecules. In this regard interaction between, homo- and heterodimerisation or oligomerisation of proteins can also be determined on the basis of the FRET phenomenon. It is possible to measure conformational changes to proteins if the donor and acceptor fluorophores are coupled to the same molecule. Similarly, within molecular biology, the principle of FRET in the quantification of nucleic acids using realtime PCR (realtime polymerase chain reaction) is implemented in practice via the use of special probes.

In this connection it is possible to utilise FRET to quantify nucleic acids with the use of ‘LightCycler®’ probes, which are special hydrolysis probes, wherein various oligonucleotides that are each labelled with a donor or acceptor, bind side-by-side to the target sequence of a nucleic acid molecule and thus bring the donor and acceptor close enough to one another for FRET are employed as probes for the quantification of PCR products.

A further commonly-used option for FRET consists of the application of ‘TagMan®’ probes, which are special hydrolysis probes, wherein the probe is labelled at one end with an acceptor or quencher and at the other end with a donor or reporter fluorescence dye. With intact hydrolysis probes the fluorescence of the donor fluorophore is inhibited by the quencher as a result of radiationless energy transfer (FRET). During a PCR cycle the probe hybridises with the complementary DNA strand whilst donor fluorescence initially remains suppressed. Taq polymerase forms the 5′ end of the probe during the PCR cycles. The fluorescence of the donor is now no longer quenched by the quencher and can be measured.

The use of ‘molecular beacons’ as probes is a further option for realtime quantification of PCR products with utilisation of FRET. Molecular beacons are oligonucleotides that are coupled both to a donor and to a quencher or acceptor. The nucleotides at the 5′ end of the probe are complementary to the nucleotides at the 3′ end so a loop-like secondary structure characteristic for molecular beacons can be formed. In this state, known as stem loop, the donor exhibits no fluorescence as a result of its proximity to the acceptor. By attachment of the loop region to a complementary DNA sequence during a PCR cycle, the distance between donor and acceptor is increased in such a way that donor fluorescence can be observed.

The structure and functional principle of molecular beacons are described in detail by Tyagi and Kramen (1996): “Molecular beacons: probes that fluoresce upon hybridization” in Nature Biotechnology 14, 303-308. They are also the subject of U.S. Pat. No. 5,925,517, which is hereby referenced in its entirety for the purposes of disclosure regarding the structure and function of molecular beacons.

5′-(2-aminoethyl)-aminonaphthalin-1-sulphonic acid (EDANS) at the 5′ end is known as a fluorophore/quencher pair and 4-(4′-dimethyl amino)phenylazo benzoic acid (DABCYL) at the 3′ end is known as a quencher. The use of DABCYL in combination with fluorescein at the 5′ end is also suitable (G. Leone, von Schijndel H., van Gemen B., Kramen R. F. and Schön D. (1998) “Molecular Beacon Probes Combined with Amplification by NASBA Enable Homogeneous Real-time Detection of RNA” in: Nucleic Acids Research 26, 9, 2150-2155).

Since, owing to the quenching caused by FRET, molecular beacons emit no, or very little fluorescence radiation or cause a spectral shift with use of two fluorophores and thus remove the signal from the measurement range when the sample sequence is not hybridised to the target sequence, molecular beacons have recently proven to be particularly effective as probes in detection systems in which the washing out of excess probes is either undesired or not possible. This applies particularly in the case of realtime PCR or in the case of detection of nucleic acids in living cells (Liu X., Farmerie W., Schuster S., Tan W. (2000): “Molecular beacons for DNA Biosensors with a Micrometer to Submicrometer Dimension” in: Analytical Biochemistry 283, 56-63).

Even in complex biological systems such as living cells however, the non-specific fluorescence, for example of cellular constituents, has proven to be a substantial systematic source of errors that generally considerably reduces the validity and specificity of the detection methods themselves, even with use of the known molecular beacons or FRET probes. For example haemoglobins or aromatic hydrocarbons exhibit non-specific fluorescence with excitation by UV light and this fluorescence may manifest itself negatively as background radiation. The actual measurement signal is therefore superposed. This background radiation is also referred to as autofluorescence of the sample environment or sample medium. Within the scope of the FRET process, particularly with regard to its molecular biological application, there is thus a substantial need to increase the efficiency of the respective FRET probes. Not only the specific selection and adjustment of the dyes used is of considerable significance in this instance, but also the configuration of the probe in view of the spatial spacing between the dyes. As described above, the FRET phenomenon thus only occurs with very small spacings between donor and acceptor and decreases with the sixth power of the distance between the dyes or fluorophores. In view of this in particular, (not always entirely) successful attempts have previously been made in the prior art to provide efficient probes, in particular molecular beacons. The probes of the prior art do not always have an optimal emission spectrum, which particularly affects differentiation of the emission spectrum from autofluorescence of a measurement sample or sample medium. In addition, the emission times often are not optimal in this regard since they generally subside just a few nanoseconds after excitation so the emission of the FRET probe has to be detected within the scope of a time-resolved measurement at the same time as the excitation signal, which is unfavourable with regard to the detection and evaluation of the signal. In addition, there is not always optimal FRET with the FRET probes known from the prior art, so quenching of the donor is not always optimal.

German patent DE 102 59 677 B4, which can be traced back to the Applicant, relates to a method for identifying and quantifying microorganisms in a sample, the target nucleic acid being amplified following isolation of the microorganisms and transfer of the isolated microorganisms to a PCR vessel, and the target nucleic acid being determined using a special probe comprising at least two probes forming a FRET pair. The FRET probe used only emits an identifiable signal following hybridisation of the probe with a target nucleotide, which signal has an extended lifetime compared to the fluorescence emission of the sample environment so the measurement signal can be distinguished from the autofluorescence of the sample environment by a time-resolved fluorescence measurement. In this connection, with regard to the FRET probe used, a special FRET pair based on EuTTAPhen and Cy5 is applied previously. A combination of this type of fluorescence dyes also poses a considerable improvement over the prior art with regard to emission behaviour, but the properties in this respect are not always optimal. In particular, the probes described in the German patent are a ‘single-colour assay’ which makes it possible to establish in particular, albeit within a limited scope, the distance between the corresponding fluorescence dyes.

### BRIEF SUMMARY OF THE INVENTION

In view of this the object of the present invention is to provide a FRET complex or FRET system that is adapted for the aforementioned applications and, in particular, avoids at least some of the drawbacks of the prior art or at least mitigates these.

In particular, an object of the present invention is to provide a FRET complex or a FRET system that is adapted, for example, for molecular biological analysis methods for qualitative and quantitative determination of target molecules such as nucleic acid molecules, for example within the scope of realtime PCR, and for determination of structural changes, for example in proteins. In particular a FRET complex or FRET system with improved emission behaviour, particularly with regard to emission duration and width of the emission bands, is provided in this instance, it being possible in particular to differentiate or discriminate from the autofluorescence of a sample. In addition, improved qualitative establishment of the distance between the dye complexes and therefore of mutual reaction with the target molecule or target structure is made possible based on the FRET complex or FRET system according to the invention.

In order to solve the problem detailed above, the present invention (in accordance with a first aspect) proposes the use of at least two different fluorophores for configuring a fluorescence resonance energy transfer pair (FRET pair). Further advantageous configurations of the use according to the invention are the subject of the relevant claims.

A further subject-matter of the present invention (in accordance with a second aspect) is the fluorescence resonance energy transfer complex (FRET complex) in accordance with the invention. Further advantageous configurations of the FRET complex according to the invention are the subject of the relevant claims.

In addition, a further subject-matter of the present invention (according to a third aspect) is the fluorescence resonance energy transfer system (FRET system) in accordance with the invention. Further advantageous configurations of this aspect of the invention are the subject-matter of the relevant claims.

A further subject-matter of the present invention (according to a fourth aspect) is the use in accordance with the invention of the FRET complex or FRET system according to the invention for detection of an event in a sample. Further advantageous configurations of the use in accordance with the invention according to the fourth aspect are the subject of the relevant claims.

A further subject-matter of the present invention (according to a fifth aspect) is the use in accordance with the invention of the FRET complex or FRET system according to the invention for interaction with a target molecule or a target structure. Further advantageous configurations of the use in accordance with the invention according to this aspect are the subject of the relevant claims.

Furthermore, a further subject-matter of the present invention (according to a sixth aspect) is the use in accordance with the invention of the FRET complex or FRET system according to the invention as a dye for labelling purposes or the like. Further advantageous configurations of the use in accordance with the invention according to this aspect are the subject of the relevant claims.

Furthermore, a further subject-matter of the present invention (according to a seventh and eighth aspect) is the use in accordance with the invention of the FRET complex or FRET system according to the invention in, or as biosensors or else in, or as (bio)probes, particularly FRET probes. Further advantageous configurations of the use in accordance with the invention according to this aspect are the subject of the relevant claim.

Lastly, a yet further subject-matter of the present invention (according to a ninth aspect) is a fluorescence resonance energy transfer pair (FRET pair).

### BRIEF DESCRIPTION OF THE DRAWINGS

FIG. 1 shows a FRET complex or FRET system according to the invention that comprises a terbium complex (Tb) as a donor fluorophore and a europium complex (Eu) as an acceptor fluorophore where the two fluorophores are interconnected by an organic radical (spacer or linker), which can be nucleotide sequence that comprises complementary hybridised nucleotides on its ends.

FIG. 2 shows an optical spectrum of the FRET complex or FRET system according to the invention illustrated in FIG. 3. As a result of the effective energy transfer of the Tb complex to the Eu complex, there is significantly reduced or only very little emission through the Tb complex.

FIG. 3 shows a FRET complex or FRET system according to the invention based on a Tb complex as fluorophore (A) and based on a europium complex as fluorophore (B), the complexes being connected by an organic radical in the form of a linear hydrocarbon chain via urethane bridges.

FIG. 4 shows a europium complex that is used within the scope of the present invention as fluorophore (B) and comprises four hydroxy picolinates as ligands.

FIG. 5 shows a terbium complex that is used within the scope of the present invention as fluorophore (A) and comprises four hydroxy picolinates as ligands.

### DETAILED DESCRIPTION OF THE INVENTION

In order to avoid repetitions it goes without saying that configurations, embodiments, advantages and the like described hereinafter with regard to merely one aspect of the invention do, of course, also apply accordingly to the further aspects of the invention.

The applicant has now surprisingly found that the problem described above can be solved by using at least two special, different fluorophores, as defined hereinafter, for configuration of a fluorescence resonance energy transfer pair (also referred to synonymously as a FRET pair). The FRET pair comprises a first fluorophore (A) as a donor fluorophore and at least a second fluorophore (B) as an acceptor fluorophore. The use in accordance with the invention is characterised in that the first fluorophore (A) and the second fluorophore (B), independently of each other, each are configured on the basis of an organo-metal complex of rare earth elements. Within the scope of the present invention the fluorophores (A) and (B) are selected in such a way that they comprise different rare earth elements from each other.

The FRET pair configured within the scope of the use in accordance with the invention is particularly adapted for use within the scope of biochemical or molecular biological analysis methods, for example for detection of specific target molecules or target structures, particularly biomolecules such as DNA, RNA, proteins and the like in a sample, and thus effectively as a FRET probe, in this instance the FRET pair configured within the scope of the use in accordance with the invention also possibly comprising specific portions or constituents that are able to mutually react with the target molecule or target structure. In addition, the FRET pair prepared within the scope of the present invention is adapted for the ascertainment or detection of conformational changes to molecules, for example of proteins or nucleic acids. Cleaving and attachment processes of biomolecules can be examined with the FRET pair prepared in accordance with the invention. In addition, use as a dye is also possible.

Within the scope of the present invention, an efficient system or complex can be successfully prepared by specific selection of the fluorophores (A) and (B), each with different rare earth elements, and the further special configuration of the FRET system (described hereinafter), particularly with regard to the spatial arrangement of the fluorophores (A) and (B) relative to one another, said system or complex being particularly adapted within the scope of FRET analysis or FRET examination for use, in particular, in molecular biological or biochemical analyses for detection or discovery, and for analysis of spatial changes to specific target molecules or target structures.

In this instance the FRET pairs prepared within the scope of the present invention are characterised in that they comprise a defined emission spectrum with a very narrow emission band, which leads to excellent detectability, in particular since the emission spectrum of the FRET pair prepared in accordance with the invention differs significantly from the non-specific fluorescence emission of a sample environment and thus from autofluorescence in such a way that excellent discrimination of the desired measurement signal from non-specific background radiation is achieved.

This property of the FRET pairs or FRET probes according to the invention and thus likewise of the FRET complexes or FRET systems of the present invention described hereinafter is thus improved yet further since the FRET pairs prepared in accordance with the invention have a very long emission duration, i.e. the FRET pairs or FRET probes according to the invention release a temporally extended emission signal following excitation by an accordingly suitable excitation signal, which emission signal further improves detectability. The duration of the signal emission of the FRET pair according to the invention is thus several milliseconds, whilst for example the undesired interference fluorescence based on autofluorescence of the sample environment subsides within nanoseconds (for example approximately 200 nanoseconds) following termination of an excitation signal. This results in a further improvement in the differentiability or discrimination between the measurement signal on the one hand and the interference signal on the other.

A further decisive advantage of the FRET pair or FRET system according to the invention is that, in this instance, they are two-colour assays as a result of the specific selection of the fluorophores (A) and (B), which comprise different rare earth elements, said assays being adapted for qualitative and quantitative analysis of mutual reactions or structural changes and the like. In accordance with a preferred embodiment according to the invention, the FRET system in accordance with the invention can thus luminesce red with use of special terbium and europium complexes for the fluorophores (A) and (B) with a short distance between the fluorophores (A) and (B) and thus with maximal energy transfer, whilst the fluorophores exhibit yellow luminescence at a long distance. In the intermediate states, i.e. with average distances between the fluorophores, the FRET pair or FRET probe according to the invention luminesces within the orange colour range, more specifically with a corresponding colour transition from red to yellow depending on the distance between the fluorophores. This is also particularly advantageous for use of the FRET pairs according to the invention as dyes.

In view of this, the FRET pair or FRET probe and thus the FRET system or FRET complex prepared within the scope of the present invention are particularly adapted for use within the field of biochemistry or molecular biology and cell biology. In this regard the FRET pair prepared in accordance with the invention is particularly adapted for the determination of protein/protein, protein/nucleic acid and nucleic acid/nucleic acid mutual reactions, and of conformational changes and cleaving processes of, or in biomolecules, even quantitative analysis of the underlying events being possible as a result of the specific configuration as a two-colour assay. The FRET system according to the invention is particularly suitable within the scope of quantification of nucleic acids, for example within the scope of realtime PCR.

In accordance with a first aspect, the subject-matter of the present invention is thus the use of at least two different fluorophores for configuring a fluorescence resonance energy transfer pair (FRET pair), within the FRET pair at least a first fluorophore (A) acting as a donor fluorophore and at least a second fluorophore (B) acting as an acceptor fluorophore. The use according to the invention is characterised in that the first fluorophore (A) and the second fluorophore (B), independently of each other, each are configured on the basis of an organo-metal complex of rare earth elements, the fluorophores (A) and (B) comprising different rare earth elements from each other.

The underlying principle of the present invention can thus be regarded as the fact that, with regard to the FRET pair, a first fluorophore (A) is used as a donor that comprises a central atom from the group of rare earths. In addition, in accordance with the invention a second fluorophore (B) is used that similarly comprises a rare earth element as the central atom, in this instance the rare earth element being a different element with regard to the first fluorophore (A). The selective use of rare earth elements with regard to the first fluorophore (A) and the second fluorophore (B), with the provision that different elements from this group are used in the respective fluorophores, completely surprisingly results in the excellent properties of the FRET pair prepared within the scope of the use according to the invention with regard to its emission properties, such as narrow emission bands, long emission or luminescence durations, quantitative change to the emission spectrum as a function of the distance between the fluorophores (A) and (B) within the scope of a two-colour assay, and excellent FRET with a short distance between the fluorophores with optimal quenching of the donor signal.

Within the scope of the present invention it has proven to be particularly advantageous if the first fluorophore (A) and the second fluorophore (B) are selected in such a way that an energy transfer from the first fluorophore (A) to the second fluorophore (B) takes place under the influence and/or effect and/or application of an excitation energy, in particular the energy transfer from the first fluorophore (A) to the second fluorophore (B) occurring at least in a substantially radiationless manner. An energy transfer of this type takes place, in particular, with a short distance between the fluorophores within the meaning of FRET. As the distance increases, FRET decreases. With regard to the excitation energy, an electromagnetic radiation in particular with a wavelength that is optimised or oriented toward the donor can be used. This is known to the person skilled in the art per se.

The energy transfer from the first fluorophore (A) to the second fluorophore (B) may take place, for example, via dipole/dipole interactions. The presence of an energy transfer may be detected in particular by a decrease in the fluorescence of the fluorophore (A) or of the donor fluorophore and/or by an increase in the fluorescence of the second fluorophore (B) or of the acceptor fluorophore.

Fluorescence microscopes, fluorimeters or the like are non-limiting examples of detection and measurement units.

The energy transfer from the first fluorophore (A) to the second fluorophore (B) thus may be dependent on the distance between the fluorophores (A) and (B). In this connection the energy transfer, particularly the intensity and/or efficiency of the energy transfer, may increase with a decreasing spatial spacing between the fluorophores (A) and (B). In other words an increase in the spacing between the first fluorophore (A) and the second fluorophore (B) leads to a reduction in the energy transfer, accompanied by a change to the emission spectrum. For example a change of this type to the spacing between the fluorophores (A) and (B) may occur within the scope of a mutual reaction or interaction with, or attachment of the FRET pair to a target molecule or a target structure, this mutual reaction or interaction with, or attachment to the target structure causing a change in the distance, particularly an increase in the distance between the two fluorophores (A) and (B). The underlying result can be seen in a change to the emission spectrum, the donor signal increasing and/or the acceptor signal decreasing with increasing spacing. If the distance is reduced, the donor signal correspondingly decreases and the acceptor signal increases as a result of the quenching effect of the acceptor.

In this connection the energy transfer from the first fluorophore (A) to the second fluorophore (B) can thus lead to a decrease in the fluorescence of the first fluorophore (A) and to an increase in the fluorescence of the second fluorophore (B).

In accordance with the invention it is advantageous for the fluorophore (A) and the fluorophore (B) to have at least substantially parallel electronic planes of polarisation, which is particularly significant if the fluorophore (A) on the one hand and the fluorophore (B) on the other hand are fixed (for example on the same protein). A change to the FRET signal with a change in the position of the fluorophores (A) and (B) relative to one another within the scope of interaction with a protein or a conformational change to the protein itself can thus explain the change to the planes of polarisation and thus the position of the two fluorophores relative to one another.

As already explained, the fluorophore (A) and the fluorophore (B) should only be spaced apart at a short distance in order to enable an energy transfer, particularly a fluorescence resonance energy transfer, the spacing generally measuring a few nanometres. This is because the intensity of the FRET signal decreases with the sixth power of the distance between the fluorophores. In this connection the efficiency of the energy transfer is dependent on the ‘transfer radius’ of the fluorophore pair. The criterion of a short distance between fluorophore (A) and fluorophore (B) results in the FRET pair prepared within the scope of the use according to the invention possibly acting, to a certain extent, as an ‘optical nanometre scale’ and thus being adapted, for example, for qualitative and quantitative determination of the spacing between the fluorophores within the scope of molecular biological or biochemical methods in order to thus draw qualitative and quantitative conclusions regarding underlying primary events, such as interaction with a target molecule or a target structure and conformational changes.

With regard to the energy transfer, particularly fluorescence resonance energy transfer, particularly with a short spacing between the fluorophores (A) and (B), it may also be provided for the states of the first fluorophore (A) to overlap, at least in part, with the states of the second fluorophore (B).

Furthermore, the first fluorophore (A) and/or the second fluorophore (B), independently of each other, each should be and/or comprise a dye, particularly a fluorescence dye.

Within the scope of the present invention it is advantageous for the first fluorophore (A) and/or the second fluorophore (B), independently of each other, to each comprise at least one core, preferably a core, formed of a rare earth element. It is particularly preferable for the first fluorophore (A) and/or the second fluorophore (B), independently of each other, to be present in the form of a coordination complex or a coordination compound or in the form of a chelate compound, the central atom(s) being formed by the rare earth element. Within the scope of the present invention it is particularly preferable (if multi-core coordination complexes are used) for cores based on the same rare earth element to be used within a complex and thus within a fluorophore.

Particularly good results with regard to the aforementioned properties of the FRET pair prepared within the scope of the use according to the invention can be achieved if the rare earth element is selected from the group of scandium, yttrium, lanthanum, cerium, praseodymium, neodymium, promethium, samarium, europium, gadolinium, terbium, dysprosium, holmium, erbium, thulium, ytterbium, and lutetium.

Within the scope of the present invention it may particularly be provided for the rare earth element to be selected from the elements of lanthanides, particularly from the group of cerium, praseodymium, neodymium, promethium, samarium, europium, gadolinium, terbium, dysprosium, holmium, erbium, thulium, ytterbium, and lutetium.

Lanthanides are silvery, shiny, relatively soft and reactive metals that oxidise rapidly when exposed to air and become matt. In water they react more or less quickly with the release of hydrogen gas. Lanthanides are generally a total of 14 elements from the sixth period of the Periodic Table of the Elements that can be considered as a subcategory of the third subgroup. As a result of the similar structure of the valence shells, lanthanides behave chemically in a manner comparable to that of the elements in the third group of the Periodic Table of the Elements, namely scandium and yttrium. Together with these, lanthanides form the group of rare earths.

Particularly good results in terms of the FRET pair prepared within the scope of the present invention are obtained if the rare earth element is selected from the group of europium and terbium.

In this regard the applicant has completely surprisingly found that excellent properties in terms of the FRET pair are produced if the fluorophore (A) comprises terbium as a rare earth metal, particularly in the form of terbium(III).

In this connection it has likewise proven to be particularly advantageous if the fluorophore (B) comprises europium as a rare earth metal, particularly in the form of europium(III).

It is consequently particularly preferred within the scope of the present invention for the FRET pair prepared within the scope of the use according to the invention to comprise a fluorophore (A) and a fluorophore (B), the fluorophore (A) comprising terbium as a rare earth metal, particularly in the form of terbium(III), and the fluorophore (B) comprising europium as a rare earth metal, particularly in the form of europium(III). A FRET pair of this type exhibits excellent properties in terms of quenching behaviour in the presence of an energy transfer, particularly fluorescence resonance energy transfer, with a short spacing between the fluorophores (A) and (B) and defined emission behaviour with narrow emission bands and extended emission duration, in such a way that such a FRET based on terbium and europium is adapted, to a certain extent, for analytical use within the field of biochemistry or molecular biology.

With regard to the configuration of the fluorophores of the FRET pair prepared within the scope of the use according to the invention it is thus preferable, in accordance with the invention, for the rare earth elements each to be complexed in the fluorophores (A) and (B), particularly in each case with at least one ligand, particularly a complexing and/or chelating agent, especially a plurality of ligands, preferably four ligands, and/or for the rare earth elements in the fluorophores (A) and (B), independently of each other, to be bonded ionically, coordinatively and/or covalently, particularly covalently, to at least one ligand, particularly to a plurality of ligands, preferably to four ligands.

In this connection the ligand, particularly the complexing and/or chelating agent, should be formed in a multidentate, particularly bidentate manner. In this connection the ligand may assume a plurality of coordinative, ionic or covalent bonds with the core or central atom based on a rare earth metal. In principle it is possible within the scope of the present invention for the same or different ligands to be used with regard to the fluorophores (A) and (B). The same or different ligands can also be used within the respective fluorophore (A) or (B) itself.

According to a preferred embodiment in accordance with the invention the ligand, particularly the complexing and/or chelating agent, is picolinic acid, picolinates and/or derivatives thereof, particularly substituted derivatives, preferably hydroxy derivatives.

In order to enable a bond between the fluorophores and at least a further molecule or organic radical, the ligand, particularly the complexing and/or chelating agent, particularly the picolinic acid, picolinates and/or derivatives thereof, should comprise substituents and/or functional groups in particular for preferably covalent bonding and/or coupling of preferably organic molecules. In this regard the substituents and/or functional groups should be selected from the group of amino, carboxylate, isocyanate, thioisocyanate, epoxy, thiol and hydroxyl groups. ‘Joining points’ for further molecules or for at least one organic radical are thus provided on the respective fluorophores (A) or (B). For example, as mentioned below, the organic molecule may be a unit that is complementary to a target molecule or to a target structure or interactable therewith.

According to a particularly preferred embodiment, the substituent or functional group should be a hydroxyl group.

Furthermore, within the scope of the present invention it is particularly advantageous for the ligand, particularly the complexing and/or chelating agent, to be hydroxypicolinic acid and/or hydroxypicolinate. In this connection at least one of the ligands should be formed by the aforementioned compounds, particularly if a covalent bond or coupling of preferably organic molecules to the fluorophores (A) and/or (B) is intended.

Furthermore, within the scope of the present invention it is particularly preferable for the fluorophore (A) to be an organo-metal complex according to the formula of FIG. 5.

In addition it may be provided in accordance with the invention for the fluorophore (A) to be selected from tetra(4-hydroxypyridin-2-carboxylato)terbium(III), tris-(pyridin-2-carboxylato)(4-hydroxypyridin-2-carboxylato)terbium(III), bis(pyridin-2-carboxylato)-bis(4-hydroxypyridin-2-carboxylato)terbium(III), (pyridin-2-carboxylato)-tris(4-hydroxypyridin-2-carboxylato)terbium(III) and/or derivatives thereof.

With regard to the compounds for the fluorophore (A), the corresponding salts, particularly sodium salts, can also be used in particular.

In addition, the fluorophore (A) may be a compound of the general formula

[Tbₓ(Pic)_{y}(Pic-Y)_{z}]^{(4-3x)-}

where, in this formula
Tb is terbium(III),
Pic is picolinate,
Y is a functional group, in particular selected from the group of amino, carboxylate, isocyanate, thioisocyanate, epoxy, thiol and hydroxyl groups, preferably a hydroxyl group,
x is an integer from 1 to 4, particularly 1 or 2, preferably 1, and
y and z are each an integer from 0 to 4 where y+z=4.

With regard to the fluorophore (B), this fluorophore (B) may be an organo-metal complex according to formula 4 of FIG. 4.

In this connection it is particularly preferable in accordance with the invention for the fluorophore (A) to be an organo-metal complex according to the formula of FIG. 5 and for the fluorophore (B) to be an organo-metal complex according to the formula of FIG. 4.

Furthermore, the fluorophore (B) may be selected from tetra(4-hydroxypyridin-2-carboxylato)europium(III), tris(pyridin-2-carboxylato)(4-hydroxypyridin-2-carboxylato)europium(III), bis(pyridin-2-carboxylato)-bis(4-hydroxypyridin-2-carboxylato)europium(III), (pyridin-2-carboxylato)-tris(4-hydroxypyridin-2-carboxylato)europium(III) and/or derivatives thereof.

With regard to the compounds concerning the fluorophore (B), for example the corresponding salts, particularly sodium salts, can also be used.

With reference to the fluorophore (B), a compound of the general formula

[Eu_{x′}(Pic′)_{y′}(Pic′-Y′)_{z′}]^{(4-3x′)-}

may be provided, where, in this formula
Eu is europium(III),
Pic′ is picolinate,
Y′ is a functional group, in particular selected from the group of amino, carboxylate, isocyanate, thioisocyanate, epoxy, thiol and hydroxyl groups, preferably a hydroxyl group,
x′ is an integer from 1 to 4, particularly 1 or 2, preferably 1, and
y′ and z′ are each an integer from 0 to 4 where y′+z′=4.

For example the fluorophores (A) and (B) of a FRET pair or FRET probe can be configured or arranged for example by coupling or attachment of the fluorophores to a molecule, particularly a biomolecule, for example to a protein or the like, a fluorescence resonance energy transfer taking place, as described before, with a short spatial spacing and the emission spectrum being changed with increasing spacing between the fluorophores (A) and (B) and a decrease in the energy transfer, which may be used for example as a scale or indicator for a conformational change to the molecule.

Similarly and in particular for the configuration of a FRET pair or FRET probe, it is also possible within the scope of the present invention for the fluorophore (A) and the fluorophore (B) to be coupled and/or connected to one another via a particularly divalent and/or two-bond organic radical, particularly a linker and/or spacer.

In this regard a molecule or molecule complex is thus produced that comprises the fluorophore (A) and the fluorophore (B) as well as the radical. The purposeful selection of the organic radical and adjustment to the special fluorophores (A) and (B) can thus optimise or tailor the FRET pair or FRET probe inasmuch as, with a short spacing between the fluorophores (A) and (B), an optimal fluorescence resonance energy transfer can take place in terms of maximum quenching of the donor signal and, with increasing spacing, a well-defined change to the emission spectrum with narrow bands and long emission durations is obtained.

Within the scope of the present invention the organic radical should thus be selected in such a way that a fluorescence resonance energy transfer can take place between the fluorophore (A) and the fluorophore (B). This applies particularly when there is a short spacing between the fluorophores (A) and (B), for example if there is no mutual reaction with the target molecule or target structure. The fluorescence resonance energy transfer can thus be adjusted or tailored individually by the targeted selection of the spacer, a small spacer, i.e. in particular a molecule with a short chain length, leading to a short spacing between the fluorophores (A) and (B), which is accompanied by a high fluorescence resonance energy transfer. However, a long radical or spacer may also lead to a short spacing between (A) and (B), for example via folding and/or attachment or hybridisation processes, as is the case for example with a molecular beacon. Owing to the individual adjustment of the spacing between the fluorophores (A) and (B) by the organic radical, the emission spectrum can thus be adjusted specifically, in particular emission spectra with specific wavelengths or emission bands also being produced with FRET, which is of considerable significance particularly within the scope of production or use as dyes, since the colour properties so to speak can be adjusted individually by the selective adjustment of the spacing between the fluorophores (A) and (B).

On the whole, the spacing between the fluorophores (A) and (B) and thus the fluorescence resonance energy transfer and therefore the emission wavelength of the FRET pair can thus be adjusted or tailored by the length of the organic radical, or by the structure, spatial arrangement or configuration thereof. The organic radical may be connected or coupled to the fluorophores in such a way, for example, that the organic radical can be bonded to the respective substituents and/or to the respective functional group of the complexing agent and/or of the ligand, particularly as defined above.

Within the scope of the present invention it is preferable for the organic radical to be bonded preferably covalently to the fluorophore (A) and/or the fluorophore (B), in particular the organic radical preferably being connected and/or coupled covalently to the functional groups of the ligand(s) of the fluorophore (A) and/or (B), and/or in particular the organic radical comprising functional groups capable of bonding and/or coupling, preferably covalently, to the functional groups of the ligand(s).

The organic radical particularly connects or couples to the functional groups in such a way that the fluorophores (A) and (B), independently of each other, are bonded or coupled to the organic radical, preferably terminally, and thus, to a certain extent, to the respective end of the organic radical.

In this connection the organic radical can be joined covalently to the fluorophore (A) and/or the fluorophore (B), for example with the formation of terminal (poly)urethane groups, particularly with regard to the organic radical.

In accordance with the invention it may likewise be provided for the respective fluorophores, independently of each other, each to comprise an organic radical, which may be the case for example if the FRET pair prepared within the scope of the use according to the invention is used as a FRET probe of the hybridisation probe type. In this instance the organic radical may be, for example, an oligonucleotide. A preferably terminal bond or coupling of the fluorophores (A) and (B) to an organic radical is present, for example, if the FRET pair prepared in accordance with the use according to the invention is provided or is to be used as a FRET probe of the hydrolysis probe type.

With regard to the organic radical in general, this organic radical may thus be a radical that is able to mutually react with a target molecule and/or a target structure. The organic radical may be such that it is a radical that can interact with a target molecule and/or a target structure.

Within the scope of the present invention it may be provided for an interaction and/or mutual reaction between the organic radical and the target molecule and/or target structure to lead to a change, in particular a reduction in the fluorescence resonance energy transfer from the fluorophore (A) to the fluorophore (B).

This is achieved in particular in that, owing to the interaction or mutual reaction, the spacing between the first fluorophore (A) and the second fluorophore (B) is increased in such a way that the energy transfer between the fluorophores decreases and the emission behaviour is thus changed. The interaction or mutual reaction can thus be detected by the ascertainment of (a change in) the emission spectrum of the sample.

For example the organic radical may be, or may comprise a nucleotide sequence, in particular a DNA or RNA sequence, preferably an oligonucleotide.

For example this may be the case when the FRET pair prepared within the scope of the use according to the invention is used as a hybridisation probe, a hydrolysis probe and/or a molecular beacon.

Particularly when the FRET pair prepared in accordance with the use according to the invention is used as a molecular beacon it may be provided for the nucleotide sequence to comprise, particularly at the 5′ end and 3′ end thereof, complementary nucleotide sequence portions, particularly in such a way that when there is no mutual reaction with a target molecule and/or a target structure there is hybridisation of the complementary nucleotide sequence portions. Self-hybridisation of the ends of the nucleotide sequence occurs, to a certain extent, as a result of the complementary end portions of the nucleotide sequence in such a way that the structure described above, which is characteristic of molecular beacons, is present in the form of a stem loop. In this state the fluorophore (A) exhibits no fluorescence or reduced fluorescence as a result of its short distance from the fluorophore (B). If there is attachment of the loop region to a complementary DNA sequence as a target molecule or target structure, for example during a PCR cycle, the distance between the fluorophore (A) and the fluorophore (B) is increased in such a way that, in this instance, a fluorescence or change in fluorescence caused by reduced fluorescence resonance energy transfer results with regard to the fluorophore (A) and this can be observed or detected using devices or methods known to the person skilled in the art, the change to the emission spectrum being accompanied by an increase in donor fluorescence and a decrease in acceptor fluorescence.

As mentioned above, the FRET pair prepared in accordance with the use according to the invention may be, for example, a molecular beacon (MB). In this connection it is provided for the fluorophore (A) and the fluorophore (B) to form a molecular beacon (MB) by particularly covalent bonding to a nucleotide sequence with complementary nucleotide sequence portions, particularly on the 5′ ends and 3′ ends thereof.

Similarly, it is also possible within the scope of the present invention for the organic radical to be or to comprise a peptide and/or an amino acid sequence, in particular an oligopeptide.

In this instance it may be, for example, an enzyme or a defined amino acid sequence oriented toward a respective target structure or target molecule. Likewise, it may also be a peptide or an amino acid sequence of which the spatial structure itself is to be ascertained.

In addition, it may also be provided within the scope of the present invention for the organic radical to be, or to comprise a saturated or unsaturated hydrocarbon radical, preferably an unsaturated, branched or linear hydrocarbon radical.

In this connection it is particularly advantageous for the hydrocarbon radical to have a chain length of at least 2 carbon atoms, in particular 2 to 20 carbon atoms, preferably 2 to 15 carbon atoms, preferably 2 to 12 carbon atoms.

As mentioned above, in this instance also the length of the hydrocarbon radical should be selected in view of the energy transfer, particularly the fluorescence resonance energy transfer to be set between the fluorophore (A) and the fluorophore (B).

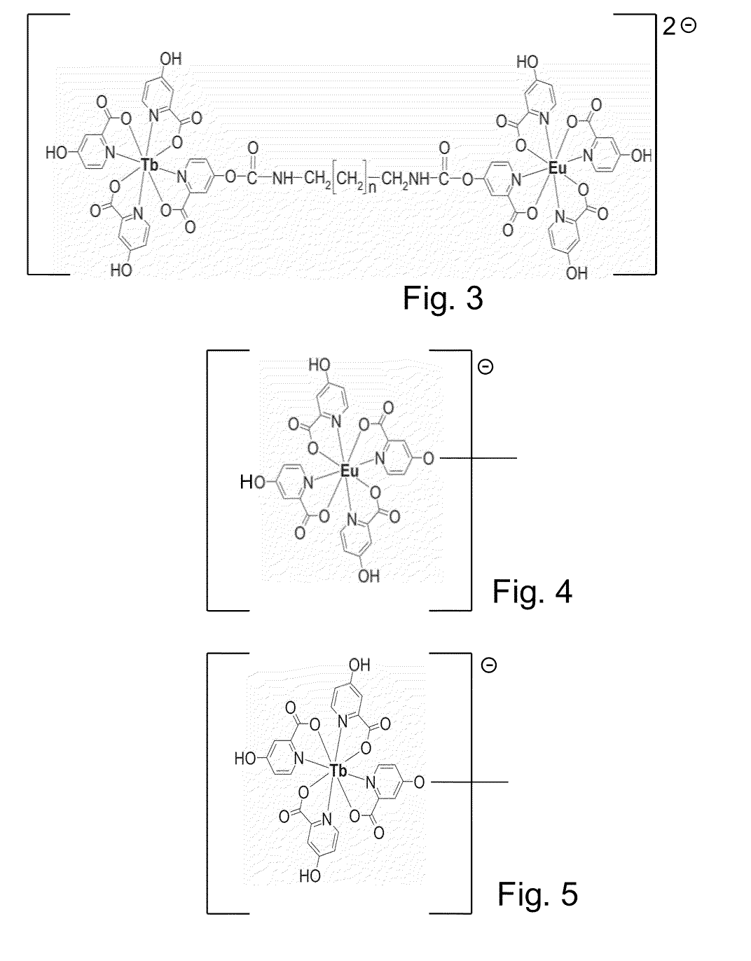

In accordance with a particularly preferred embodiment according to the invention the fluorophore (A) and the fluorophore (B) form, together with an organic radical, the complex according to the formula from FIG. 3, where in this formula n is an integer in the range from 1 to 20, particularly 2 to 15, preferably 2 to 12.

In accordance with the formulae of FIGS. 3 to 5, the fluorophore (A) is formed by a terbium complex with four hydroxy picolinates and the fluorophore (B) is formed by a europium complex with four hydroxy picolinates. The fluorophores are interconnected by a hydrocarbon radical at terminal urethane bonds.

The organic radical may also be a mixed molecule that contains a nucleotide sequence and/or an amino acid sequence and/or a hydrocarbon radical.

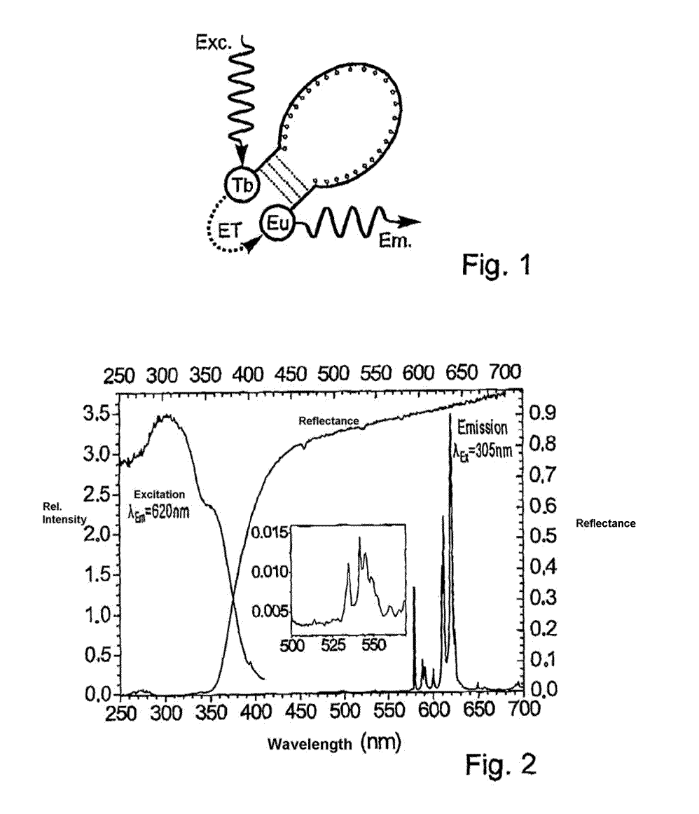

The present invention will now be illustrated further in a purely exemplary manner with reference to the figures, although the invention is not limited hereto, in which:
FIG. 1 shows a FRET complex or FRET system according to the invention that comprises a terbium complex (Tb) as a donor fluorophore and a europium complex (Eu) as an acceptor fluorophore. The two fluorophores are interconnected by an organic radical (spacer or linker), in this instance said organic radical particularly possibly being a nucleotide sequence that comprises complementary hybridised nucleotides on its ends. The unhybridised part of the nucleotide sequence forms a loop region. The complex illustrated in FIG. 1 particularly represents a molecular beacon (MB) that is present in a closed state, in the present case a particularly radiationless energy transfer (ET) of the terbium complex to the europium complex taking place upon radiation of an excitation energy and being accompanied by a specific emission (Em.) through the europium complex.
FIG. 2 shows an optical spectrum of the FRET complex or FRET system according to the invention illustrated in FIG. 3. As a result of the effective energy transfer of the Tb complex to the Eu complex, there is significantly reduced or only very little emission through the Tb complex.
FIG. 3 shows a FRET complex or FRET system according to the invention based on a Tb complex as fluorophore (A) and based on a europium complex as fluorophore (B), the complexes being connected by an organic radical in the form of a linear hydrocarbon chain via urethane bridges.
FIG. 4 shows a europium complex that is used within the scope of the present invention as fluorophore (B) and comprises four hydroxy picolinates as ligands.
FIG. 5 shows a terbium complex that is used within the scope of the present invention as fluorophore (A) and comprises four hydroxy picolinates as ligands.

The present invention further relates (in accordance with a second aspect) to a fluorescence resonance energy transfer complex (FRET complex) comprising at least a first fluorophore (A) as a donor fluorophore and at least a second fluorophore (B) as an acceptor fluorophore, the fluorophore (A) and the fluorophore (B) being coupled and/or connected to one another via a particularly divalent and/or two-bond organic radical, in particular a linker and/or spacer. The FRET complex according to the invention is characterised in that the first fluorophore (A) and the second fluorophore (B), independently of each other, each are configured on the basis of an organo-metal complex of rare earth elements, the fluorophores (A) and (B) comprising different rare earth elements from each other.

In accordance with a preferred embodiment of the present invention the FRET complex according to the invention may correspond to the general formula

DF-S-AF

where, in this formula
DF is a donor fluorophore, particularly the fluorophore (A),
AF is an acceptor fluorophore, particularly the fluorophore (B), and
S is a particularly divalent and/or two-bond organic radical, particularly a linker and/or spacer.

For further configurations with regard to the FRET complex according to the invention, reference can be made to the configurations regarding the use according to the invention described above, these configurations applying likewise with regard to the FRET complex according to the invention.

For example with regard to analytical methods, the FRET complex according to the invention may be configured for example in the form of a hybridisation probe, a hydrolysis probe or a molecular beacon.

Furthermore, the invention further relates (in accordance with a third aspect) to a fluorescence resonance energy transfer system (FRET system) comprising at least a first fluorophore (A) as a donor fluorophore and at least a second fluorophore (B) as an acceptor fluorophore. The FRET system according to the invention is characterised in that the first fluorophore (A) and the second fluorophore (B), independently of each other, each are configured on the basis of an organometal complex of rare earth elements, wherein the fluorophores (A) and (B) comprise different rare earth elements from each other.

In accordance with this aspect of the present invention the FRET system may be present for example in the form of the respective complexes DF′-M and AF′-M′, DF′ being a donor fluorophore, particularly the fluorophore (A), AF′ being an acceptor fluorophore, particularly the fluorophore (B), and M and M′, independently of each other, being an organic radical, the respective radicals M and M′ being bonded, particularly covalently, to the donor fluorophore or to the acceptor fluorophore.

In accordance with this embodiment according to the invention both the fluorophore (A) and the fluorophore (B) thus each comprise an organic radical that is adapted, for example, in each case for interaction and/or mutual reaction with, and/or attachment to a target structure or target molecule, for example a nucleotide sequence or an amino acid sequence. In this regard reference can be made to the above definition of the organic radical. In accordance with this embodiment according to the invention the FRET system according to the invention thus may be, for example, ‘hybridisation’ probes.

In accordance with this aspect of the present invention it thus may be provided for the fluorophore (A) and the fluorophore (B), independently of each other, each to comprise an organic radical, particularly as defined above.

Alternatively it may also be provided for the fluorophore (A) and the fluorophore (B) to be coupled and/or connected to one another via a particularly divalent and/or two-bond organic radical, particularly a linker and/or spacer, particularly as defined above.

For example in accordance with this embodiment a molecular beacon may, in turn, be involved.

On the whole, the FRET complexes or FRET systems according to the invention may be the hybridisation probes, hydrolysis probes and molecular beacons already described above. In this connection the energy transfer properties or parameters of the FRET complexes or FRET systems according to the invention may be adjusted by the specific selection of the fluorophores (A) and (B) and of the organic radical. The energy transfer properties thus adjusted can be used to calibrate intramolecular distances or spacings in hybridised nucleotide molecules, particularly in the form of molecular beacons, that are equipped with the special fluorophores (A) and (B) on the respective ends, the organic radical in the form of an oligonucleotide thus acting as a spacer or linker. Biochemical induction using a suitable target sequence or target structure opens the loop-shaped closed nucleotide sequence in such a way that an amended emission response is provided as a result of an amended energy transfer when the ends of the loop are separated or detached.

In particular the FRET complexes or FRET systems according to the invention comprise well-defined emission spectra, the emission duration lying within the millisecond range after excitation by an incorporated signal, and additional discrimination from non-specific autofluorescence thus being possible. These two factors make it possible to dispense with complex laser and evaluation systems for analysis of the measurement signals. In addition, optimal quenching is provided with a small spacing between the fluorophores, which further improves signal quality.

Furthermore, with regard to the FRET complex or FRET system according to the invention a very specific and meaningful realtime detection can be carried out within the scope of the corresponding use as biosensors, for example within the scope of amplification of target nucleic acids, with a lower detection threshold, particularly within the scope of realtime PCR in a compact and simple Thermocycler.

The use of the FRET complex or FRET system according to the invention is not limited to the coupling of nucleic acids. It is thus also possible for the FRET complex or FRET system according to the invention to be present in the form of a protein as an organic radical that bonds the fluorophores (A) and (B) and, in the case of cleaving of the protein or else owing to a conformational change, for example in the case of bonding to a ligand, for there to be a change to the spatial spacing between the fluorophores (A) and (B) in such a way that there is a change to the emission spectrum. In this instance the fluorescence emission of the donor fluorophore and thus of the fluorophore (A) can also be ascertained in particular. The FRET complex or FRET system according to the invention can thus also be used in protein or peptide analysis.

In addition, the FRET complexes or FRET systems according to the invention can also be used in isothermal amplification methods, such as nucleic acid sequence based amplification (NASBA). The FRET complexes or FRET systems according to the invention can also be used in array technology. In addition, attachment or connection of the FRET complexes or FRET systems according to the invention to a solid substrate is possible, for example for use in biochips or biosensors. In addition, a use of the FRET complexes or FRET systems according to the invention in the form of aptamers can likewise be considered. Overall, the very large field of application of medical, molecular biological and biochemical analysis and diagnosis is thus opened to the FRET complexes or FRET systems according to the invention.

For further embodiments relating to the third aspect of the present invention, reference can be made to the embodiments above regarding the first and second aspects of the present invention, which can be applied accordingly.

A further subject-matter of the invention (according to a fourth aspect) is the use in accordance with the invention of the FRET complex or FRET system defined above according to the invention for detection of an event in a sample.

The term ‘event’, as used within the scope of the present invention, is to be interpreted very broadly. For example, the event thus may be an interaction and/or reaction and/or hybridisation between the FRET complex or FRET system and a DNA sequence or RNA sequence or an oligonucleotide, for example within the scope of nucleic acid detection. In this instance the organic radical of the FRET complex or FRET system according to the invention preferably comprises or consists of a nucleotide sequence. Similarly, the term ‘event’ also includes an interaction and/or mutual reaction and/or attachment of the FRET complex or FRET system according to the invention with/to a (poly)peptide or enzyme. Similarly, the event may also be the cleaving or else the bonding of a peptide from/to a ligand. In this instance the organic radical of the FRET complex or FRET system according to the invention should preferably be, or comprise an amino acid sequence or an oligopeptide or polypeptide. Similarly, the event may also be a structural change, for example a configurational or conformational change to a protein or the like. The event is thus generally, to a certain extent, an activity within a test sample that particularly leads to a change in the spatial arrangement or spacing of the fluorophore (A) and the fluorophore (B) relative to one another, whereby in particular, as a result of, or owing to this change, a preferably discriminating detection of a changed measurement signal, particularly a change in the emission spectrum of the FRET complex or FRET system is present, it being possible to trace this change back to the increasing signal of the fluorophore (A) acting as a donor and the decreasing signal of the acceptor, caused by the increase in the spatial spacing between the fluorophores (A) and (B) with a reduction in the fluorescence resonance energy transfer.

With regard to the use according to the invention in accordance with this aspect of the present invention, the FRET complex and/or the FRET system can thus undergo a change to its emission spectrum when the event occurs and/or as a result of the event, particularly under the influence and/or effect and/or application of an excitation energy. In addition, the FRET complex and/or FRET system may emit a detectable signal when the event occurs and/or as a result of the event, particularly under the influence and/or effect and/or application of an excitation energy.

In accordance with the invention it may thus be provided for the relative arrangement of the fluorophores (A) and (B) to be changed by, or as a result of the event, particularly in such a way that the emission spectrum of the FRET complex and/or FRET system is changed and/or a detectable signal is emitted.

In accordance with the invention it may also be provided for the change to the emission spectrum and/or the detectable signal to be temporally delayed relative to the event, and/or for the change to the emission spectrum and/or the detectable signal to take place after the event has occurred.

Furthermore, the change to the emission spectrum and/or the detectable signal can be ascertained by a time-resolved fluorescence measurement. In this regard it should be possible to distinguish the change to the emission spectrum or the detectable signal from autofluorescence.

In this regard it is advantageous for the detectable signal of the FRET complex and/or of the FRET system to be extended by a factor of 2, particularly by a factor of 5, especially by a factor of 10, and preferably by a factor of 100 compared to the autofluorescence.

Lastly, as mentioned above, the event may be a mutual reaction and/or interaction and/or hybridisation of the FRET complex and/or FRET system with a target molecule and/or a target structure, particularly a nucleotide sequence, preferably a DNA or RNA sequence, or a peptide and/or an amino acid sequence. In addition, the event may be a spatial change, particularly a configurational and/or conformational change to the FRET complex and/or the FRET system.

A further subject-matter of the present invention (in accordance with a fifth aspect) is the use in accordance with the invention of the FRET complex according to the invention, as defined above, for interaction with a target molecule and/or a target structure and/or for detection and/or identification and/or for determination of a target molecule and/or a target structure, particularly by mutual reaction of the FRET complex and/or of the FRET system on the one hand with the target molecule and/or the target structure on the other.

The target molecule or target structure thus may be a nucleotide sequence, preferably a DNA or RNA sequence, a (poly)peptide and/or an amino acid sequence, particularly an enzyme or an antibody.

Similarly, it is also possible within the scope of the present invention to detect or identify viruses and microorganisms (particularly bacteria) on the basis of the FRET complex or FRET system according to the invention. In this connection the corresponding organic radical of the FRET system or FRET complex according to the invention should be configured in such a way that interaction is possible between viruses, microorganisms (particularly bacteria), antibodies and the like and specific target structures of the aforementioned biological systems.

For example, the FRET complex or FRET system according to the invention can be used or implemented for the identification or quantification of microorganisms, particularly of microorganisms in foodstuffs, for example accumulation of the microorganisms occurring first. By way of non-limiting example this may take place with a carrier coated with a ligand, the ligand being specific to the microorganisms to be identified or quantified. In addition, accumulation may also take place via microparticles coated with antibodies. After optional lysis and purification, the corresponding target nucleic acids can then be amplified using the FRET complexes or FRET probes according to the invention.

The present invention similarly comprises a method for the identification or detection of target oligonucleotides in a sample environment, comprising the following steps: in a first step a target nucleic acid is incubated using the FRET complex or FRET system according to the invention with subsequent radiation of the incubation formation and detection of the fluorescence emission, particularly by time-resolved fluorescence measurement. In this connection, realtime PCR for example can be used to amplify the target nucleic acids. Similarly, in this regard the target nucleic acid or FRET complex or FRET system according to the invention can be bonded to a carrier as a FRET probe.

Lastly, the present invention relates (in accordance with a sixth aspect) to the use of the FRET complex or FRET system according to the invention as defined above as a dye, in particular a fluorescence dye, in particular for labelling purposes.

As a result of the special structure accompanied by the special fluorophores based on rare earth metals, the fluorophores can produce specific colorations in a specific manner as a result of the selection of the fluorophores and the adjustment of the spacing between the fluorophores, in such a way that the FRET complexes or FRET systems according to the invention are also particularly adapted for use as dyes.

With regard to dyes, defined colorations are possible within a narrow spectrum as a result of the precise and narrow emission bands.

Overall, the principle underlying the present invention of utilisation of the energy transfer or fluorescence resonance energy transfer between complexes of terbium on the one hand and complexes of europium on the other can be used as a valuable medium within the scope of studying structure, (fine) tuning of emission and improvement of the properties of biosensors or dye complexes. This is a clear advantage of the present invention.

Furthermore, the present invention relates (in accordance with a seventh aspect) to the use of the FRET complex or FRET system according to the invention defined above in or as biosensors or else in or as probes, particularly FRET probes. Lastly, the present invention relates (in accordance with an eighth aspect) to a fluorescence resonance energy transfer pair (FRET pair) that comprises at least two different fluorophores, within the FRET pair at least a first fluorophore (A) acting as a donor fluorophore and at least a second fluorophore (B) acting as an acceptor fluorophore, the first fluorophore (A) and the second fluorophore (B), independently of each other, each being configured on the basis of an organo-metal complex of rare earth elements, the fluorophores (A) and (B) comprising different rare earth elements from each other.

For further embodiments regarding the fifth to ninth aspects of the present invention, reference can be made to the above embodiments relating to the further aspects of the present invention, which can be applied accordingly.

Further configurations, modifications and variations of the present invention will be readily recognisable and reproducible by the person skilled in the art upon reading the description without departing from the scope of the present invention.

The present invention will now be illustrated with reference to the following practical examples without any limitation of the present invention thereto.

### PRACTICAL EXAMPLES

### Ligand Modification:

3-aminopicolinic acid or 3-hydroxypicolinic acid is dissolved in distilled water, the corresponding amount of 1M NaOH solution is neutralised and crystallised out as 3-aminopicolinic acid sodium salt or 3-hydroxypicolinic acid sodium salt.

0.3222 g of 3-aminopicolinic acid sodium salt or 3-hydroxypicolinic acid sodium salt (2 mmol) is dried under vacuum (2·10⁻² mbar) at 110° C. for 3 hours, dissolved in approximately 30 ml of dried DMF and, for example, 0.162 ml of hexamethylene diisocyanate (HDI or OCN—(CH₂)₆—CNO) or, for example, 0.269 ml od dodecamethylene diisocyanate (DMDI or OCN—(CH₂)₁₂—CNO), (1 mmol) is added in an inert gas atmosphere. The reaction is also carried out in the presence of inert gas at 90° C. for 24 hours or 70 hours with reaction of sodium-3-hydroxypicolinate. DMF is removed under vacuum at 90° C.

The products have the compositions NaOOC—C₅H₄N—NH—CO—NH—(CH₂)ₙ—NH—CO—NH—C₅H₄N—COONa (n=6 for the product with HDI and n=12 for the product with DMDI) with the reaction of sodium 3-aminopicolinate, and NaOOC—C₅H₄N—O—CO—NH—(CH₂)ₙ—NH—CO—O—C₅H₄N—COONa with the reaction of sodium-3-hydroxypicolinate.

With comparison modification of the ligands with monoisocyanates (phenylisocyanate or butylisocyanate) and butylisothiocyanate, 2 mmol of the isocyanate reagent are accordingly prepared for the same reaction.

### Synthesis of the Complex Compounds

### Tetrapicolinate Complexes:

0.4350 g of picolinic acid sodium salt (3 mmol) and 0.1600 g of 3-aminopicolinic acid sodium salt (or 3-hydroxypicolinic acid sodium salt) (1 mmol) are dissolved in approx. 30 ml of DMSO with heating (with synthesis of the complexes with only one type of ligand, 4 mmol of the corresponding sodium salt are accordingly prepared). 0.3664 g of EuCl₃.6H₂O (or else 0.3730 g of TbCl₃.6H₂O or else 0.18324 g of EuCl₃.6H₂O and 0.1865 g of TbCl₃.6H₂O for the mixed complexes) (1 mmol) are dissolved in a little DMSO and the ligand solution is added. The mixture is left to react for 2 hours at 90° C., then DMSO is distilled off at the same temperature under vacuum. The beige-coloured powder thus obtained is boiled in THF under reflux for 1 hour, filtered and dried under vacuum at 80° C. for one hour.

With synthesis of the complex compounds with the modified ligands, 0.5 mmol of the ligand modified with the diisocyanate (or 1 mmol of the ligand modified with the isocyanate or isothiocyanate) is accordingly prepared instead of 1 mmol of the 3-aminopicolinic acid sodium salt.

As a result of the poor solubility of the DMDI-modified ligand, the isocyanates are modified with the rare earth complexes: 0.35 mmol of the complex NaEuₓTb₍₁₋ₓ₎(Pic)₃(Pic-Y) (x=0 to 1, Pic=picolinate and Pic-Y=3-aminopicolinate or 3-hydroxypicolinate) is dried under vacuum (2·10⁻² mbar) at 110° C. for 3 hours, dissolved in approx. 20 ml of DMSO, and 0.094 ml of DMDI (0.088 mmol) is added in an inert gas atmosphere. The reaction is carried out in the presence of argon at 90° C. for 24 hours (or 70 hours with the reaction of 3-hydroxypicolinates). DMSO is distilled off under vacuum at the same temperature. The beige-coloured powder now obtained is boiled in THF under reflex for 1 hour, filtered and dried under vacuum at 80° C. for one hour.

### Tripicolinate Complexes:

0.2900 g of picolinic acid sodium salt (2 mmol) and 0.1600 g of 3-aminopicolinic acid sodium salt (or 3-hydroxypicolinic acid sodium salt) (1 mmol) are dissolved in approx. 30 ml of DMF with heating (with synthesis of the complexes with only one type of ligand, 3 mmol of the corresponding sodium salt are prepared). 0.3664 g of EuCl₃.6H₂O (or else 0.3730 g of TbCl₃.6H₂O or else 0.1832 g of EuCl₃.6H₂O and 0.1865 g of TbCl₃.6H₂O for the mixed complexes) (1 mmol) are dissolved in a little DMF and the ligand solution is added. The precipitate produced is filtered, washed with ethanol and dried under vacuum at 50° C. for one hour.

With synthesis of the complex compounds with the modified ligand, 0.5 mmol of the ligand modified with the diisocyanate (or 1 mmol of the ligand modified with the isocyanate or isothiocyanate) is accordingly prepared instead of 1 mmol of the 3-aminopicolinic acid sodium salt.

With synthesis of the complex compounds with exclusively modified ligands, for example Eu₂[NaOOC—C₅H₄N—O—CO—NH—(CH₂)ₙ—NH—CO—O—C₅H₄N—COO]₃, the precipitate is produced only with heating to 90 to 100° C.

### Labelling of Isocyanate-Modified DNA:

The complex NaEuₓTb₍₁₋ₓ₎(Pic)₃(Pic-Y) (for example x=0 for labelling with merely a green luminous complex; x=1 for labelling with merely a red luminous complex; x=0.5 for labelling with green and red luminous complexes; Pic=picolinate; Pic-Y=3-aminopicolinate or 3-hydroxypicolinate) is dried under vacuum (2·10⁻² mbar) at 110° C. for 3 hours, dissolved in DMSO and the appropriate amount of the DNA solution in dried DMSO (molar ratio Pic-Y:NCO=1:1) is added in an inert gas atmosphere. The reaction is carried out in the presence of argon at 90° C. for 24 hours (or 70 hours with the reaction of 3-hydroxypicolinates). If necessary the DMSO can be distilled off under vacuum at the same temperature.

### Labelling of Isothiocyanate-Modified DNA:

The complex NaEuₓTb₍₁₋ₓ₎(Pic)₃(Pic-Y) (for example x=0 for labelling with merely a green luminous complex; x=1 for labelling with merely a red luminous complex; x=0.5 for labelling with green and red luminous complexes; Pic=picolinate; Pic-Y=3-aminopicolinate or 3-hydroxypicolinate) is dissolved in DMSO and the appropriate amount of the DNA solution in water, DMSO or a DMSO/water solution (molar ratio Pic-Y:NCS=1:1) is added. The reaction is carried out for 24 hours (or 70 hours with the reaction of 3-hydroxypicolinates). If necessary the DMSO or water can be distilled off under vacuum at the same temperature.

1-60. (canceled)

61. A method of configuring a fluorescence resonance energy transfer pair (FRET pair), the method comprising the step of using at least two different fluorophores for configuring the FRET pair, wherein within the FRET pair at least one first fluorophore (A) acts as a donor fluorophore and at least one second fluorophore (B) acts as an acceptor fluorophore, wherein the first fluorophore (A) and the second fluorophore (B), independently of each other, each are configured on the basis of an organo-metal complex of rare earth elements, the fluorophores (A) and (B) comprising different rare earth elements from each other.

62. The method according to claim 61, wherein the first fluorophore (A) and the second fluorophore (B) are selected in such a way that an energy transfer from the first fluorophore (A) to the second fluorophore (B) takes place under the influence of an excitation energy, wherein the energy transfer from the first fluorophore (A) to the second fluorophore (B) is dependent on the spacing between the fluorophores (A) and (B), the energy transfer increasing with decreasing spatial spacing between the fluorophores (A) and (B).

63. The method according to claim 61, wherein the first fluorophore (A) and the second fluorophore (B), independently of each other, each comprise at least one core formed of a rare earth element, wherein the rare earth element is selected from the group of scandium, yttrium, lanthanum, cerium, praseodymium, neodymium, promethium, samarium, europium, gadolinium, terbium, dysprosium, holmium, erbium, thulium, ytterbium, and lutetium.

64. The method according to claim 61, wherein the fluorophore (A) comprises terbium as a rare earth metal and wherein the fluorophore (B) comprises europium as a rare earth metal.

65. A fluorescence resonance energy transfer complex (FRET complex), comprising: at least a first fluorophore (A) as a donor fluorophore and at least a second fluorophore (B) as an acceptor fluorophore, the fluorophore (A) and the fluorophore (B) being coupled or bonded to one another via an organic radical, wherein the first fluorophore (A) and the second fluorophore (B), independently of each other, each are configured on the basis of an organo-metal complex of rare earth elements, the fluorophores (A) and (B) comprising different rare earth elements from each other.

66. The florescence resonance energy transfer complex according to claim 65, wherein the FRET complex corresponds to the general formula
DF-S-AF
wherein, in this formula,
DF is the donor fluorophore (A),
AF is the acceptor fluorophore (B), and
S is a divalent organic radical in the form of a linker or spacer.

67. The florescence resonance energy transfer complex according to claim 65, wherein the first fluorophore (A) and the second fluorophore (B) are selected in such a way that an energy transfer from the first fluorophore (A) to the second fluorophore (B) takes place under the influence of an excitation energy, the energy transfer from the first fluorophore (A) to the second fluorophore (B) occurring at least in a substantially radiationless manner, wherein the energy transfer from the first fluorophore (A) to the second fluorophore (B) is dependent on the spacing between the fluorophores (A) and (B), the energy transfer increasing with decreasing spatial spacing between the fluorophores (A) and (B).

68. The florescence resonance energy transfer complex according to claim 65, wherein the first fluorophore (A) and the second fluorophore (B), independently of each other, each comprise at least one core formed of a rare earth element selected from the group of scandium, yttrium, lanthanum, cerium, praseodymium, neodymium, promethium, samarium, europium, gadolinium, terbium, dysprosium, holmium, erbium, thulium, ytterbium, and lutetium.

69. The florescence resonance energy transfer complex according to claim 65, wherein the fluorophore (A) comprises terbium as a rare earth metal and wherein the fluorophore (B) comprises europium as a rare earth metal.

70. The florescence resonance energy transfer complex according to claim 65, wherein the rare earth elements in the fluorophores (A) and (B), independently of each other, are bonded to a plurality of ligands.

71. The florescence resonance energy transfer complex according to claim 65, wherein the fluorophore (A) is an organo-metal complex according to the formula of FIG. 5.

72. The florescence resonance energy transfer complex according to claim 65, wherein the fluorophore (A) is selected from tetra(4-hydroxypyridin-2-carboxylato)terbium(III), tris-(pyridin-2-carboxylato)(4-hydroxypyridin-2-carboxylato)terbium(III), bis(pyridin-2-carboxylato)-bis(4-hydroxypyridin-2-carboxylato)terbium(III), (pyridin-2-carboxylato)-tris(4-hydroxypyridin-2-carboxylato)terbium(III) and/or derivatives thereof.

73. The florescence resonance energy transfer complex according to claim 65, wherein the fluorophore (A) is a compound of the general formula
[Tbₓ(Pic)_{y}(Pic-Y)_{z}]^{(4-3x)-}
where, in this formula.
Tb is terbium(III),
Pic is picolinate,
Y is a functional group, in particular selected from the group of amino, carboxylate, isocyanate, thioisocyanate, epoxy, thiol and hydroxyl groups, preferably a hydroxyl group,
x is an integer from 1 to 4, particularly 1 or 2, preferably 1, and
y and z are each an integer from 0 to 4 where y+z=4.

74. The florescence resonance energy transfer complex according to claim 65, wherein the fluorophore (B) is an organo-metal complex according to the formula of FIG. 4.

75. The florescence resonance energy transfer complex according to claim 65, wherein the fluorophore (B) is selected from tetra(4-hydroxypyridin-2-carboxylato)-europium(III), tris-(pyridin-2-carboxylato)(4-hydroxypyridin-2-carboxylato)europium(III), bis(pyridin-2-carboxylato)-bis(4-hydroxypyridin-2-carboxylato)europium(III), (pyridin-2-carboxylato)-tris(4-hydroxypyridin-2-carboxylato)europium(III) and/or derivatives thereof.

76. The florescence resonance energy transfer complex according to claim 65, wherein the fluorophore (B) is a compound of the general formula
[Eu_{x′}(Pic′)_{y′}(Pic′-Y′)_{z′}]^{(4-3x′)-}
where, in this formula,
Eu is europium(III),
Pic′ is picolinate,
Y′ is a functional group, in particular selected from the group of amino, carboxylate, isocyanate, thioisocyanate, epoxy, thiol and hydroxyl groups, preferably a hydroxyl group,
x′ is an integer from 1 to 4, particularly 1 or 2, preferably 1, and
y′ and z′ are each an integer from 0 to 4 where y′+z′=4

77. The florescence resonance energy transfer complex according to claim 65, wherein the fluorophore (A) and the fluorophore (B) are coupled to one another via a divalent organic radical.

78. A method for detection of an event in a sample, the method comprising the step of using a fluorescence resonance energy transfer complex (FRET complex) according to claim 65.

79. The method according to claim 78, wherein the FRET complex undergoes a change as to its emission spectrum when the event occurs or as a result of the event and wherein the FRET complex and/or FRET system emits a detectable signal when the event occurs or as a result of the event.

80. The method according to claim 78, wherein the relative arrangement of the fluorophores (A) and (B) is changed by or as a result of the event.

81. A method for interaction with a target molecule or a target structure, the method comprising the step of using a fluorescence resonance energy transfer complex (FRET complex) according to claim 65 in such a way that a mutual reaction between the FRET complex on the one hand and the target molecule and the target molecule or structure on the other hand occurs.

82. A fluorescence resonance energy transfer pair (FRET pair), comprising at least two different fluorophores, within the FRET pair at least one first fluorophore (A) acting as a donor fluorophore and at least one second fluorophore (B) acting as an acceptor fluorophore, wherein the first fluorophore (A) and the second fluorophore (B), independently of each other, each are configured on the basis of an organo-metal complex of rare earth elements, the fluorophores (A) and (B) comprising different rare earth elements from each other.
